(19) United States
(12) Patent Application Publication
(10) Pub. No.: US 2003/0212355 A1
(43) Pub. Date: Nov. 13, 2003

(54) Multi-purpose back brace

(76) Inventor: Billy J. Shilling, Silver Spring, MD (US)

Correspondence Address: Office Of Counsel, Bldg 112T, Naval Undersea Warfare Center, Division, Newport, 1176 Howell Street, Newport, RI, 02841-1708, US

(21) Appl. No.: 10/143,172

(22) Filed: May 7, 2002

(51) Int. Cl. A61F 5/00

(52) U.S. Cl. 602/19

(57) ABSTRACT

A lumbar support device that includes a belt circumferentially positioned around the waist of a user. There is also a pair of straps. Each of these straps has opposed ends. These ends are positionable in a first position in which one of these opposed ends is attached to the rear section of the belt. The strap extends over one of the shoulders of the user and is then attached to the other side of the belt at its other opposed end. These straps are both removable from the first position to an alternate second position. In this alternative second position, one of the opposed ends is attached to the waist belt and the strap extends from the belt adjacent one of the legs of the user to overlap one of the knees. The strap then extends back to the belt and is attached to the belt at the other of its opposed ends.

### STATEMENT OF GOVERNMENT INTEREST

The invention described herein may be manufactured and used by or for the Government of the United States of America for governmental purposes without the payment of any royalties thereon or therefor.

### CROSS REFERENCE TO OTHER PATENT APPLICATIONS

Not applicable.

### BACKGROUND OF THE INVENTION

(1) Field of the Invention

The present invention relates to medical devices and more particularly to back braces.

(2) Brief Description of the Prior Art

Wide safety belts are now worn around the midriff of nearly all people who perform some type of labor requiring bending or lifting on a routine basis. However, these devices provide no back support or are even uncomfortable while worn seated. Individuals with back problems may require support or protection from further injury while performing tasks standing or when seated with no backrest or one that is inappropriate. Frequently, personnel of various occupations are required to carry or lift large loads to remote work areas or in the field and then operate or use this equipment. The use of equipment in various areas of operations may not afford many seats with backrests or proper back support. In these working conditions, heavy work involving lifting is performed followed by periods of seated activity or rest where a back brace may be most useful.

The prior art suggests a number of approaches to solving this problem. U.S. Pat. No. 5,176,131 to Vote et al., for example, discloses a back support for providing abdominal and lumbosacral support with a waistband of substantially unstretchable construction. An elastic band is operatively connected to the outer surface of the waistband. The waistband has a generally V shape, wherein the waistband resists riding up on a wearer. The elastic band is preferably releasably connected to the outer surface of the waistband. The support may also have a tool belt operatively connected thereto.

U.S. Pat. No. 5,375,279 to Toso discloses a combination back support for use during stretching and/or sitting with the legs extended or not extended. The device includes a substantially rectangular lumber back support with a multiplicity of pairs of inelastic straps used in a first configuration to engage the knees of a user and with the addition of a strap extension used to engage the feet of a user. When the user is one of the various seated positions, the straps are connected to form loops with are fitted over either the feet or knees of the user. As the user applies pressure with the feet or knees, the lumbar rectangular support is pulled tight around the user's lower back, providing ample support.

U.S. Pat. No. 5,399,151 to Smith discloses a unisize type lifting belt containing basically three elements: the underlying wrap assembly with expandable back panel and overlapping closure ends; suspenders with four adjustable ends to secure to the underlying wrap assembly at varying positions in front and at two positions varying vertically in the rear; and expandable side pull portions, which have a pair of elastic band portions and removably secured end tabs is disclosed. The expandable side pulls are normally applied by snugly securing the side pulls to the underlying wrap assembly prior to lifting.

U.S. Pat. No. 5,499,965 to Sanchez discloses a shaped lifting belt and method in which the shape is provided by deepening the central lumbar panel in the rear to a depth of at lest an additional half of the depth of the side wraps, providing the lumbar panel with expandable elements, and then separating the ends of the wrap from the lumbar panel by shape panels, which are stretchable and positioned to engage the upper rear portion of the hips. The method of the invention requires the wearer to first engage the lifting belt assembly and secure the suspenders in the forward desirable position. Thereafter, the shape straps are secured to the inner portion of the wrap ends. The wrap ends are then secured to a central portion of the body followed by substantially encircling the same with the side pulls. Where coloring is desired, the suspender assembly may be of a different color than the belt itself, and optionally a removable side pull may be color coded or otherwise decorated to suit the application.

U.S. Pat. No. 5,503,620 to Danzger discloses a back support belt comprised of a primary support belt including fasteners for fastening the same generally at eh frontal area of the waist of the wearer, and a secondary tensioning belt comprised of fasteners for fastening the same around the primary support belt generally at the frontal area of the waist of the wearer is provided, and includes back support belt color tensioning indicators on the secondary tensioning belt, which are visible only from the rear and side areas of the waist of the wearer, and thus not by the wearer, and which are operable to indicate both when the back support belt is properly tensioned around the waist of the wearer, and when the back support belt is not properly tensioned around the waist of the wearer, and when the back support belt is not properly tensioned around the waist of the wearer. The belt support belt color tensioning indicators are operable to indicate proper and improper tensioning of the support belt independently of the relationship between the size of the support belt and the waist size of the wearer to thus be virtually foolproof in operation. For use in instances wherein the wearer of the back support belt is forced to work in the absence of anyone qualified to observe the color tensioning indicators on the secondary tensioning belt, the back support belt will further include an additional color tensioning indicator taking the form of non-obscurable alignment marks formed on one of the primary support belt fasteners and co-operable with one of the secondary tensioning belt fasteners to indicate proper back support belt tensioning. The additional color-tensioning indicator is not, however, operable independently of the relationship between the size of the support belt and the waist size of the wearer, and is thus not foolproof in operation.

U.S. Pat. No. 5,643,184 to Toso discloses a back support for engaging the lower back region of a user, including a pair of inelastic straps and/or a combination of inelastic and elastic straps structured to engage the knees and the feet of a user. When the user is in a seated position and requires additional back support, the straps are connected to the feet and knees to exert a force on the back support for supporting the user's lower back.

U.S. Pat. No. 5,656,021 to Greengarg discloses various combinations of a detachable back belt, an apron and a lifting belt, which is a direct body-engaging member with ends that are closed underneath an apron. The lifting belt has vertical stays and an elastic body portion with belt loops to prevent the same from riding up the torso of the user. The apron is formed with the traditional lower pocket sections and can be secured tot eh suspenders, which are an extension of the lifting belt. The front of the apron is provided with a loop-hook engaging section in the shape of a transverse strap. Finally, a detachable back belt secures to the rear portion of the underlying lifting belt and wraps around the same to finalize the securement by overlapping the already assembled overlapping end portions of the lifting belt and securing the ends to the apron transverse strap. A lifting belt hook central engaging member of the detachable belt engages the back of the lifting belt.

U.S. Pat. No. 5,776,087 to Nelson et al. discloses a back brace having an at least partially elastic waist belt, a substantially non-elastic rear waist belt portion and a pair of arms extending from the rear portion and a pair of arms extending from the rear portion and having over lapping interconnecting end portions for holding the belt in place around the waist of a human body. Each of the arms carries a pocket, and each of the pockets is adapted to carry a still, flexible stay. A pair of non-elastic shoulder straps each having one end anchored by the rear member of the back brace or belt and another end adjustable anchored by a strap connector which is in turn connected to a non-elastic strap support mounted on each arm. Each of the pockets and its related insert has a curved upper portion specifically designed to adapt to the curvature of the lower rib of the body.

U.S. Pat. No. 6,083,183 to Yang discloses a waistband device having a rectangular cushion, a first connecting band disposed on a first lateral of the rectangular cushion, a second connecting band disposed on a second lateral of the rectangular cushion, a first belt connected to the first connecting band, a first keeper disposed on the first belt, a first soft pad disposed on the first belt, a make fastener disposed on a distal end of the first belt, a first loop hole defined by the first belt, a second keeper disposed on the second belt, a second soft pad disposed on the second belt, a female fastener disposed on a distal end of the second belt, and a second loop hole defined by the second belt.

The above prior art, however, reveals a dilemma for users who need back support but alternate between lifting and being seated in areas with no or inadequate back support. Safety belts provide back support while standing or working and provide protection from injury while lifting, but can not be worn or used for back support while being seated. Several devices have been devised to provide back support while being seated, but most are not readily portable and provide no support or protection while lifting or working.

A need still exists, however, for a support device which provides support or protection from back injury either while standing, lifting or when seated.

### SUMMARY OF THE INVENTION

It is an object of the present invention to provide a back support device that quickly, easily and conveniently provides support for a user's back in either a standing or sitting position.

These and other objects are met by the present invention that is a multi-purpose back brace (MPBB), which is an all-purpose back support brace. This support device provides support and protection from injury while the user is performing lifting or manual labor. The support device functions as a safety belt, and the device will provide adjustable back support while seated, even if no backrest is available such as on a stool or even sated on base ground.

Also encompassed by this invention is a belt a circumferentially positioned around the waist of the user such that said belt has a front section and a rear section. There are also a pair of straps wherein each of the straps has opposed ends and is positionable in a first position wherein one of the opposed ends is attached to the rear section of the belt and each of the straps extends over one of the user's shoulders and is then attached to the other opposed end. Each of the straps is removable from this first position to an alternate second position. In this second position one of the opposed end is attached to the waist belt and the strap extends from the belt adjacent one of the legs of the user to overlap one of the knees and to then be attached to the belt at the other of its opposed ends.

### BRIEF DESCRIPTION OF THE DRAWINGS

Other objects, features and advantages of the present invention will become apparent upon reference to the following description of the preferred embodiments and to the drawings, wherein corresponding reference characters indicate corresponding parts in the drawings and wherein:

FIG. 1 is a front elevational view of the back support device of the present invention as is worn by a user in a standing position;

FIG. 2 is a rear elevational view of the back support device of the present invention as is worn by a user in a standing position;

FIG. 3 is a side elevational view of the back support device of the present invention as is worn by a user in a sitting position with no backrest on a stool; and

FIG. 4 is a top plan view of the back support device of the present invention as is worn by a user in a sitting position with no backrest on a stool.

### DESCRIPTION OF THE PREFERRED EMBODIMENT

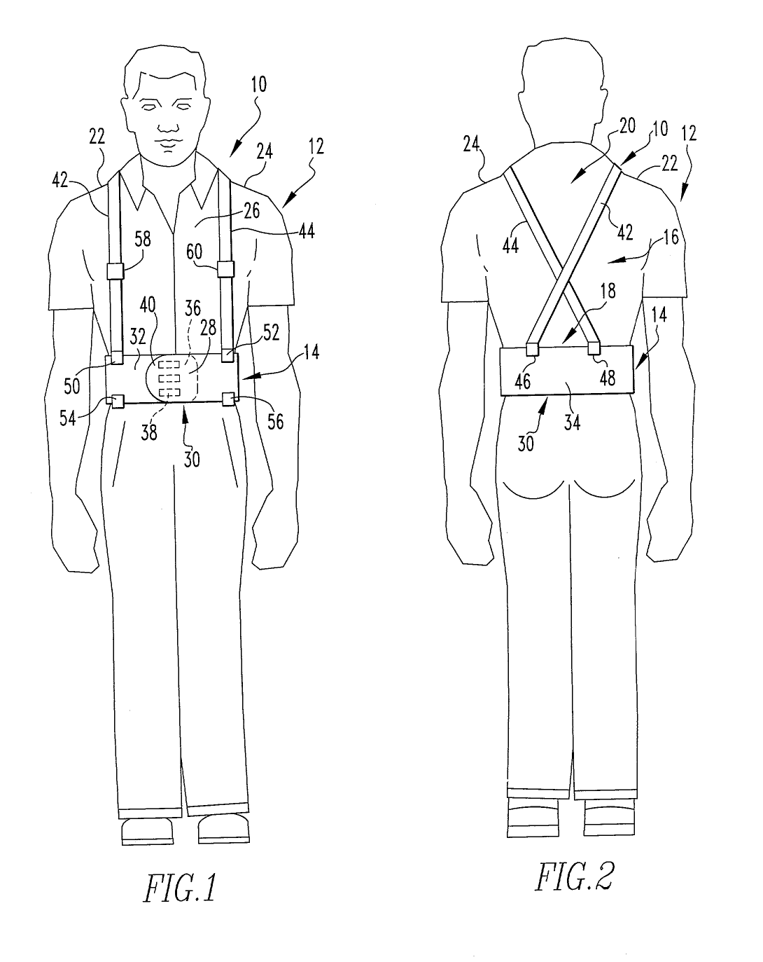

Referring to FIGS. 1 and 2, the device 10 is worn by user 12. This user 12 has a waist 14 and a back 16 with a lumbar region 18 and an upper back area 20. User 12 also has shoulders 22 and 24 chest 26 and abdomen 28. Device 10 includes a belt 30. This belt 30 has a front section 32 and a rear section 34. Front section 32 of belt 30 is closeable by means of a lower closure flap 36 with VELCRO hooks 38 and an upper flap 40. The device 10 also includes a first strap 42 and a second strap 44. First strap 42 is removably attached to belt 30 by an upper rear attachment point 46, which will preferably be comprised of VELCRO hooks. The second strap 44 will be attached to the upper rear attachment point 48. The first strap 42 and second strap 44 cross and extend upwardly over the upper back region 20 of the user 12. First strap 42 and the second strap 44 then overlap the user 12 shoulders 22 and 24 respectively. The first strap 42 and second strap 44 then extend downwardly from the shoulders 22 and 24 over the chest 26 and abdomen 28 of the user 12 and can then be attached to the front section 32 of belt 30 at upper front attachment points 50 and 52 respectively. Directly beneath the upper front attachment points 50 and 52 on belt 30 there are respectively lower front attachment points 54 and 56. On the first strap 42 there is a slip ring 58 to adjust the size of strap 42. Similarly on the second strap 44 there is a slip ring 60 to adjust the size of strap 44.

The wide belt providing back support can be worn in the desired position above the hips and below the chest or rib cage and covers the full circumference if the lower abdomen. The parts of the wide belt are continuous around the back of the wearer and have overlapping end portions that meet and close in front of the wearer. One or more strips of VELCRO material fill the full width of the belt in the overlapping region. In this way, the belt can be adjusted to snugly and comfortably fit the wearer's waist. Straps attach on the backside of the wide belt and cross over before they pass over the shoulders of the wearer. The straps can then go straight down and meet the wide belt, or they can be crossed the same as in the back if desired. VELCRO material attaches both straps to the front and the back of the belt. Both straps are necessary to ensure that the wide belt can be maintained at the desired height above the hips and below the chest while it is worn. Both straps have rectangular slip rings over the VELCRO material attachment pads in the front of the wearer. Each strap passes through the slip ring on the top and the strap is doubled for a length of run like a suspender, but for each strap the far end terminates with a catch or clasp that is also attached to and adjustable on the strap passing down the front of the wearer making a full and closed loop through the slip ring.

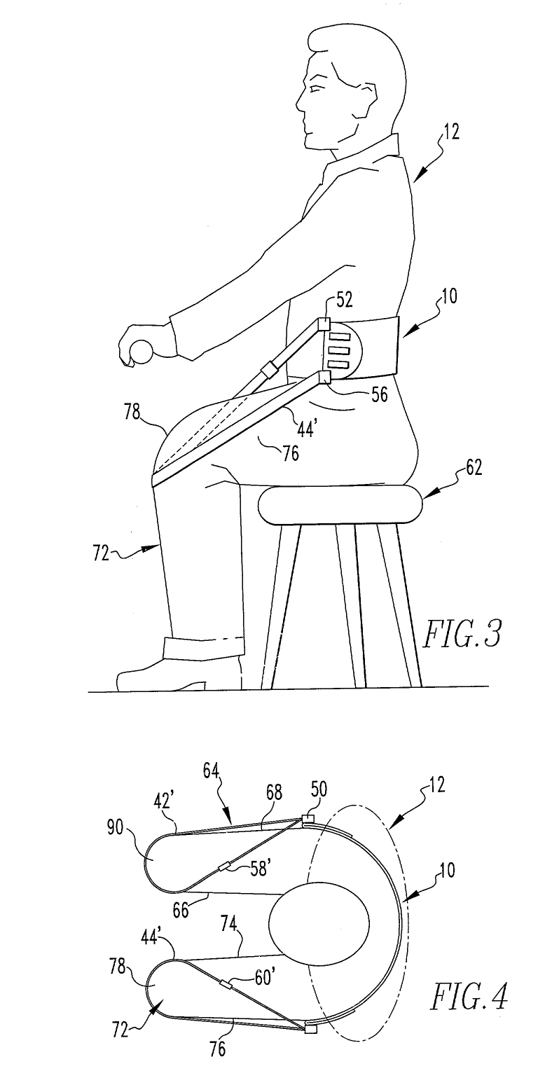

Referring to FIGS. 3 and 4, the user 12 is shown in a seated position with no backrest on a stool 62. In these drawings, the leg 64 of user 12 is shown wherein the leg has an inner section 66 and an outer section 68 as well as a knee 70. The user's 12 other leg 72 is also shown wherein this leg has an inner section 74 and outer section 76 and a knee 78. In this second position, the alternate position of the first strap 42′ is shown with its slip ring 58′. One end of first strap 42′ is removably attached to the lower front attachment point 54 from where it extends outwardly along the outer section 68 of leg 64 and wraps around knee 70, and then extends inwardly adjacent the inner section 66 of leg 64 to attach to the upper front attachment point 50 of belt 30. Similarly, second strap 44′ with slipring 60′ is removably attached at one end to the lower front attachment point 56 from where it extends outwardly along the outer section 76 of leg 72 and then wraps around knee 78 and extends inwardly along the inner section 74 of leg 72 to be then attached to the upper front attachment point 52. In this second position, the lower closure flap 36 will be disconnected from the upper closure flap 40 of belt 30. Ordinarily, the lower closure flap 36 and upper closure flap 40 will be folded rearwardly along belt 30 in opposed relation from each other.

The overlapping ends 36 and 40 of the wide waistband belt 30 have been opened and then folded back. They are held in this new position by small dots of VELCRO material about one square inch in surface area. The straps can then be repositioned and attached to the wide belt at points on the opposite side of the belt. The strap end on the lower side of the wide belt is attached at and runs along the outside of the thigh and passes round the front of the knee, below the knee cap and then returns back to the side and attaches to the top of the wide belt at position. These straps are adjustable in the same way as a pair of suspenders as when they were worn over the shoulder by using a clasp. It is desirable to have the adjustment clasp in the region where the strap passes over the top thigh for ease of adjustment by wearer. The position of waistband can be moved up or down the back to a comfortable position. The length of the leg straps and the position of the wide band can be adjusted alternately to provide the optimum position of the wide band and with the appropriate level of tension provided by the straps for comfort or needed back support while being seated. The straps in this position around the knee and attached to the top and bottom edge of the wide belt around the back of the wearer provide a firm support to the back by transferring the force generated by the wearer leaning back to the front of the leg below the knee where a counter force hold the straps in position. In so doing the wearer experiences a comfortable balance of forces between the back and the shin just below the knee.

The MPBB is an addition that is lightweight to shop and field workers which provides support and protection from back injury in nearly all circumstances working or being seated. This dual versatility is an important improvement over devices that are worn only during lifting or manual labor or only when seated for back support and can be easily worn with most existing work uniforms. The MPBB can also be incorporated or integral in work uniforms such as coveralls or specialized uniforms or protective suits that may be required when working with special or hazardous material.

Several alternatives in the use of materials for construction may be available. Flame retardant, stain resistant fabrics hold promise for military and industrial use. However, comfort cannot be ignored as its absence will discourage the use of the device. Multiple fabric and future materials, even smart materials that return to a preformed shape may be incorporated into the MPBB to provide the desired properties of shape and support that specifically match the needs of a certain wearer doing specific tasks. Certain portions or regions may be semi-rigid for support of specific body areas and the connecting regions may be of a very flexible and elastic fabric.

In an alternate embodiment, two sets of straps may be used so that the straps over the shoulders are always worn and another set of straps is used to pass around the knees and attach to the side belt when seated. This would allow the wearer to stand up and sit down frequently without the wide belt falling off or moving from its desired position across the back. The only thing that will be required to resume its use as a seated backrest is to place the straps back over the knees. The wide belt and the straps passing over the shoulder and in front of the knee could all have some type of appropriate padding to make the MPBB even more comfortable to the wearer. Alternative types of fasteners may be considered other than VELCRO to attach the straps over the shoulders and may be a requirement for the straps that pass around the knee and attach to the wide belt to provide support while being seated.

It will be appreciated that a support device has been described, which efficiently and cost effectively provides protection for a user from back injuries while the user is standing, lifting or while seated. This modification to the safety lifting belt will permit the wear to have adjustable back support while seated and also have all the support now afforded midriff safety belt wearers.

While the present invention has been described in connection with the preferred embodiments of the various figures, it is to be understood that other similar embodiments may be used or modifications and additions may be made to the described embodiment for performing the same function of the present invention without deviating therefrom. Therefore, the present invention should not be limited to any single embodiment, but rather construed in breadth and scope in accordance with the recitation of the appended claims.

What is claimed is:

1. A support device for a user having a back with a lumbar region, a waist, a pair of shoulders, and inner and outer sides and knees, said lumbar support device comprising:
a belt circumferentially positioned around the waist of the user such that said belt has a front section and a rear section; and
a pair of straps, each of said straps having opposed ends and being positionable in a first position wherein one of said opposed ends is attached to the rear section of the belt and said strap extends over one of the shoulders of the user and is then attached to the other opposed end at its other opposed end, and wherein each of said straps is removable from said first position to an alternate second position wherein one of said opposed end is attached to the belt and said straps extends from the belt adjacent one of the legs of the user to overlap one of the knees and to then be attached to the belt at the other of its opposed ends.

2. The support device of claim 1 wherein the straps are removably attached to the belt in the first position by releasable fasteners.

3. The support device of claim 1 wherein the straps are removably attached to the belt in the second position by releasable fasteners.

4. The support device of claim 1 wherein the belt has a releasable closure means.

5. The support device of claim 4 wherein the belt releasable closure means is engaged in the first position.

6. The support device claim 4 wherein the belt releasable closure means is disengaged in the second position.

7. The support device of claim 4 wherein the belt releasable closure means is positioned at the front of the belt.

8. The support device of claim 8 wherein the belt has opposed flaps adjacent to the closure means which are attached in the first position and which fold rearwardly away from each other in the second position.

9. The support device of claim 9 wherein the belt has opposed sides and the belt has opposed flaps as each releasably attached to the side of the belt in the second position.

10. The support device of claim 1 wherein the straps are substantially non-elastic.

11. The support device of claim 1 wherein the straps each have a length and means are provided to adjust said length.

12. A support device for a user having a back with a lumbar region, a waist, a pair of shoulders, and inner and outer sides and knees, said lumbar support device comprising:
a belt circumferentially positioned around the waist of the user such that said belt has a front section and a rear section;
a pair of straps, each of said straps having opposed ends and being positionable in a first position wherein one of said opposed ends is attached to the rear section of the belt at said strap extends over one of the shoulders of the user and is then attached to the other opposed end at its other opposed end, and wherein each of said straps is removable from said first position to an alternate second position wherein one of said opposed end is attached to the waist belt and said straps extends from the belt adjacent one of the legs of the user to overlap one of the knees and to then be attached to the belt at the other of its opposed ends;
a belt circumferentially positioned around the waist of the user such that said belt has a front section and a rear section and said belt has releasable closure means on the front section of said belt; and
a pair of straps wherein each of said straps having opposed ends and being positionable in a first position wherein one of said ends is attached to one of the releasable attachment means and extends there from over none of the shoulders of the user and is then attached to one of the second pair of attachment means at its other opposed end, and wherein each of said straps is removable from said first position to a second position wherein one of said opposed ends is attached to the second releasable attachment means and said strap along one side of the leg of the user and overlaps the knee of the user and then extends inwardly along the leg of the user to one of said third attachment means.

13. The support device of claim 4 wherein the belt releasable closure means is engaged in the first position.

14. The support device claim 1 where in the belt releasable closure means is disengaged in the second position.

15. The support device of claim 4 wherein the belt releasable closure means is positioned at the front of the belt.

16. The support device of claim 9 wherein the belt has opposed sides and the belt has opposed flaps as each releasably attached to the side of the belt in the second position.

17. The support device of claim 1 wherein the straps are substantially non-elastic.

18. The support device of claim 1 wherein the straps each have a length, and means are provided to adjust said length.

19. A support device for a user having a back with a lumbar region, a waist, a pair of shoulders, and inner and outer sides and knees, said lumbar support device comprising:
a belt circumferentially positioned around the waist of the user such that said belt has a front section and a rear section and said belt has releasable closure means on the front section of said belt; and
a pair of straps wherein each of said straps having opposed ends and being positionable in a first position wherein one of said ends is attached to one of the releasable attachment means and extends there from over none of the shoulders of the user and is then attached to one of the second pair of attachment means at its other opposed end, and wherein each of said straps is removable from said first position to a second position wherein one of said opposed ends is attached to the second releasable attachment means and said strap along one side of the leg of the user and overlaps the knee of the user and then extends inwardly along the leg of the user to one of said third attachment means.

20. The support device of claim 19 wherein the straps are substantially non-elastic.
